(19) United States
(12) Patent Application Publication
(10) Pub. No.: US 2023/0115120 A1
(43) Pub. Date: Apr. 13, 2023

(54) COMPOSITIONS, METHODS AND USES OF COMBINATION TREATMENTS FOR BLOOD CANCERS

(71) Applicant: University of New Brunswick, Fredericton (CA)

(72) Inventors: Alli Murugesan, Fredericton (CA); Anthony Reiman, Fredericton (CA)

(73) Assignee: University of New Brunswick, Fredericton (CA)

(21) Appl. No.: 17/911,302

(22) PCT Filed: Mar. 15, 2021
(86) PCT No.: PCT/CA2021/050339

§ 371 Date: Sep. 13, 2022

Related U.S. Application Data
(60) Provisional application No. 62/989,440, filed on Mar. 13, 2020.

(51) Int. Cl. A61K 31/216; A61K 31/454; A61P 35/00; A61P 7/00

(52) U.S. Cl. CPC A61K 31/216; A61K 31/454; A61P 35/00; A61P 7/00

(57) ABSTRACT

Methods, compositions and uses of combination treatments for cancers are disclosed including administering to the patient a therapeutically effective amount of CAPE or CAPE analog(s), and CAPE or CAPE analog(s) in combination with a histone deacetylase inhibitor or an Immunomodulatory class of compounds or Sp1 or a MYC regulating agent, wherein administration of such combinations reduces the number or growth of cancer cells or the tumor burden or tumour growth in the patient, thereby treating the patient.

### FIELD

The present disclosure relates to the field of cancer biology and agents and their combinations for treatment of cancer in general, and blood cancer in particular.

### BACKGROUND

Rigorous scientific evaluation is in pursuit of treatments for blood cancers such as myeloma, leukemia and lymphoma. Certain caffeic acid phenethyl ester (CAPE) analogs have been found to exhibit cell growth inhibitory activity on myeloma and lymphoma cell lines that are non-responsive to lenalidomide. In addition, caffeic acid phenethyl ester has been found to decrease the level of key proteins in the cereblon pathway as disclosed in PCT Patent Application No. PCT/CA2018/000198.

Histone deacetylases (HDACs) are enzymes that remove acetyl groups from the lysine residues of histones, thereby acting as gene transcription regulators (Mitsiades et al., 2003). Since changes in histone modification is a common phenomenon in several cancers including blood cancers, HDACs remain as attractive therapeutic targets. They are generally divided into several classes including class I (HDAC1, 2, 3, and HDAC8), class II (HDAC4, 5, 6, 7, 9, and HDAC10), and class IV (HDAC11). Class III includes sirtuins, SIRT 1-7 (Subramanian et al., 2010). Many pan-HDAC inhibitors have been used for the treatment of hematologic malignancies. Vorinostat and romidepsin have gained the United States (U.S.) Food and Drug Administration (FDA) approval for the treatment of patients with refractory cutaneous T-cell lymphoma (CTCL). Combination of pan-HDAC inhibitors SAHA or panobinostat (chemical formula C₂₁H₂₃N₃O₂), along with proteasome inhibitor bortezomib (BTZ) have been evaluated clinically. However, side effects such as diarrhea, fatigue, nausea, thrombocytopenia, QT-interval prolongation (Subramanian et al., 2010), etc. restricts the clinical use of the combination treatments involving HDACi. In patients receiving panobinostat, the most common side effects leading to discontinuation of therapy include diarrhea, peripheral neuropathy, fatigue, thrombocytopenia, pneumonia and cardiac toxicity (Bailey, H. et al., 2015).

The Immunomodulatory drug, lenalidomide, is a less toxic and more potent derivative of thalidomide (Marriott et al., 2002) that is used in the treatment of multiple myeloma. Lenalidomide ubiquitinates IKZF1 and IKZF3, by the CRBN-CRL4 ubiquitin ligase, leading to their degradation (Lu et al., 2014; Krönke et al., 2014). IL-2 upregulation results from the downregulation of IKZF1 and IKZF3, which increases growth and activity of T and B lymphocytes (Krönke et al., 2014). Downregulation of IKZF1 and IKZF3 is necessary and sufficient for lenalidomide's cytotoxic effects. Lenalidomide can be an effective treatment for patients with relapsed or refractory multiple myeloma who are resistant to conventional chemotherapy and thalidomide treatments (Richardson et al., 2002). Although lenalidomide may rescue cytotoxic effects in many patients that do not respond to other treatment, in a phase-3 clinical study, 40% of patients with multiple myeloma who had previously undergone other forms of antimyeloma treatment did not experience a response (Dimopoulus et al., 2007). Higher incidences of deep-vein thrombosis and pulmonary embolism in lenalidomide treatment compared to placebo were observed in this study and are serious side effects of lenalidomide.

A ubiquitous zinc finger transcription factor known as the specificity protein 1 (Sp1) binds to the GC/GT rich promoter elements and regulates the gene expression of other transcription factors such as c-myc, c-Jun, Stat1, etc. (Suske, G., 1999) involved in cancer initiation and progression. Terameprocol is a semi-synthetic small molecule that acts as a transcription inhibitor by binding to the Sp1 consensus sequence and selectively reducing the transcription of genes that have promoters controlled by Sp1 (Smolewski et al., 2008). Terameprocol has been evaluated through clinical trials for treating patients with recurrent high-grade glioma (NCT02575794). In myeloma, elevation of Sp1 expression and its DNA binding activity (Näär et al., 1998) has been established; also, Sp1 has been identified as a novel therapeutic target in MM (Fulciniti et al., 2011).

Deregulation of MYC activity has been observed in several cancers, thereby contributing to disease progression, metastasis and therapeutic resistance. Direct MYC-targeted drug development has been challenging due to structural and functional challenges associated with MYC. A few potential small molecule MYC inhibitors such as MYCi361 and related analogs have recently been evaluated (Han et al., 2019). However, small molecule inhibitors that induce epigenetic silencing, disrupt the DNA binding ability of MYC, and target proteins that modulate post-translational regulation have been employed to target MYC. Some efficacy against MYC by the inhibitors of histone deacetylases, histone methyltransferases, histone demethylases, and DNA methyltransferases, including bromodomain and extra-terminal motif (BET) bromodomains have been well documented (Allen-Petersen et al., 2019). The BET inhibitor, JQ1, has been shown to inhibit BRD4 binding at acetylated histones within the MYC promoter and enhancers, decreasing expression of MYC isoforms (Kato et al., 2016). Several BET inhibitors are being assessed, however the clinical responses have been limited, resulting in relapse (Doroshow et al., 2017).

Better treatments for cancers and blood cancers would be desirable.

### SUMMARY OF THE DISCLOSURE

The present inventors, in one aspect of the present disclosure, demonstrate in this disclosure that CAPE and its closely related analogs downregulate a cancer gene or protein target expression, wherein administration of CAPE or a CAPE analog reduces the number or growth of cancer cells or the tumor burden or tumour growth in the patient, thereby treating the patient. Upon downregulation, the levels of a target protein or gene expression is reduced whereby the function of the target protein or gene is impaired.

In another aspect, the present disclosure relates to a method for treating a patient with cancer, the method including administering to the patient a therapeutically effective amount of a compound selected from the group consisting of CAPE or a CAPE analog in combination with a downregulating agent.

The present inventors, in one aspect of the present disclosure, demonstrate in this disclosure that CAPE and its closely related analogs (i) lower histone deacetylase, particularly HDAC1 expression at both RNA and protein levels; (ii) decrease expression of cereblon pathway genes/proteins ikaros (IKZF1), aiolos (IKZF3), IRF4, and another important gene/protein target, namely MYC; and iii) decrease expression of the specificity protein 1(Sp1).

More specifically, in another aspect of the present disclosure, the data presented in this disclosure show that combining HDAC inhibitors and/or IMiDs and/or MYC targeting agents and/or Sp1 inhibitors with CAPE or potent CAPE analogs can serve as better treatment for blood cancers.

Accordingly, in one aspect, the present disclosure relates to a method for treating a patient with cancer, the method including administering to the patient a therapeutically effective amount of CAPE, having the formula:

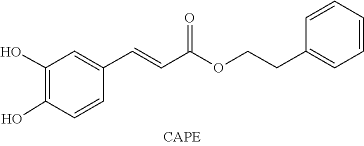 [SMILES: O=C(/C=C/c1ccc(O)c(O)c1)OCCc1ccccc1]

or a CAPE analog, in combination with a histone deacetylase inhibitor, wherein administration of CAPE or CAPE analog in combination with the histone deacetylase inhibitor reduces the number of cancer cells or the tumor burden in the patient, thereby treating the patient. Also encompassed herein is a therapeutically effective amount of CAPE or CAPE analog for use in combination with a pan-histone deacetylase inhibitor (pan-HDACi) for treating a patient with cancer, wherein administration of CAPE or CAPE analog in combination with the histone deacetylase inhibitor reduces the number of cancer cells or the tumor burden in the patient, thereby treating the patient. In a particular aspect of the method or use, the HDAC is HDAC1. In a particular aspect of the method or use, the cancer is a blood cancer. In a particular aspect of the method or use, the blood cancer is myeloma, leukemia or lymphoma. In a more particular aspect of the method or use, the CAPE analog is an analog (also referred to by the present inventors as “GL8”) according to the formula:

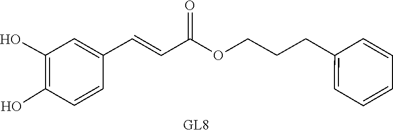 [SMILES: O=C(/C=C/c1ccc(O)c(O)c1)OCCCc1ccccc1]

In another more particular aspect of the method or use, the CAPE analog is an analog (also referred to by the present inventors as As26) according to the formula:

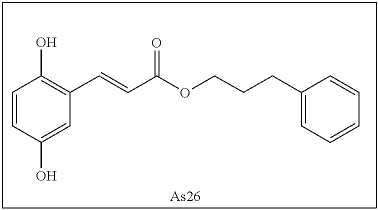

or a pharmaceutically acceptable salt thereof.

Exemplary HDACis include, without limitation, panobinostat, trichostatin A (chemical formula C₁₇H₂₂N₂O₃), entinostat (chemical formula C₂₁H₂₀N₄O₃), ricolinostat (chemical formula C₂₄H₂₇N₅O₃), romidepsin (chemical formula C₂₄H₃₆N₄O₆S₂), vorinostat (chemical formula C₁₄H₂₀N₂O₃), belinostat (chemical formula C₁₅H₁₄N₂O₄S), and LAQ824

In another aspect, the present disclosure relates to compositions for treating cancer including CAPE or CAPE analog in combination with a histone deacetylase inhibitor panobinostat.

In another aspect, the present disclosure relates to compositions for treating cancer including CAPE or CAPE analog in combination with a histone deacetylase inhibitor trichostatin A.

In another aspect, the present disclosure relates to a method for treating a patient with cancer, the method including administering to the patient a therapeutically effective amount of CAPE or CAPE analog in combination with an Sp1 inhibitor, wherein administration of CAPE or CAPE analog in combination with the Sp1 inhibitor reduces the number of cancer cells or the tumor burden in the patient, thereby treating the patient. In one aspect, the cancer is blood cancer. In another aspect, the blood cancer is selected from the group consisting of myeloma, leukemia or lymphoma. In another aspect, the cancer is brain cancer or breast cancer. In another aspect, the compound is CAPE. In another aspect, the CAPE analog is GL8. In another aspect, the CAPE analog is As26.

In another aspect, the present disclosure relates to a method for treating a patient with cancer, the method including administering to the patient a therapeutically effective amount of CAPE or CAPE analog in combination with an MYC regulating agent, wherein administration of CAPE or CAPE analog in combination with the MYC regulating agent reduces the number of cancer cells or the tumor burden in the patient, thereby treating the patient. In one aspect, the cancer is blood cancer. In another aspect, the blood cancer is selected from the group consisting of myeloma, leukemia or lymphoma. In another aspect, the compound is CAPE. In another aspect, the CAPE analog is GL8. In another aspect, the CAPE analog is As26. In another aspect, the MYC regulating agent is a BET inhibitor. In another aspect, the BET inhibitor is a small molecule inhibitor JQ1. In another aspect, the small molecule inhibitor is MYCi361 and related analogs.

In another aspect, the present disclosure relates to a composition for treating a patient with cancer including a therapeutically effective amount of CAPE or CAPE analog in combination with a therapeutically effective amount of histone deacetylase (HDAC) inhibitor. In one aspect, the HDAC is HDAC1. In another aspect, the cancer is blood cancer. In another aspect, the blood cancer is selected from the group consisting of myeloma, leukemia or lymphoma. In another aspect, the compound is CAPE. In another aspect, the CAPE analog is GL8. In another aspect, the GL8 analog is As26. In another aspect, the HDAC inhibitor is a pan-HDAC inhibitor. In another aspect, the HDAC inhibitor is panobinostat. In another aspect, the HDAC inhibitor is trichostatin A.

In another aspect, the present disclosure relates to a composition for treating a patient with cancer including a therapeutically effective amount of CAPE or CAPE analog in combination with a therapeutically effective amount of an immunomodulatory class of compounds (IMIDs). In one aspect, the IMID is lenalidomide or pomolidomide. In another aspect, the cancer is blood cancer. In another aspect, the blood cancer is selected from the group consisting of myeloma, leukemia or lymphoma. In another aspect, the compound is CAPE. In another aspect, the CAPE analog is GL8. In another aspect, the CAPE analog is As26.

In another aspect, the present disclosure relates to a composition for treating a patient with cancer including a therapeutically effective amount of CAPE or CAPE analog in combination with a therapeutically effective amount of an a Sp1 inhibitor. In another aspect, the cancer is blood cancer. In another aspect, the blood cancer is selected from the group consisting of myeloma, leukemia or lymphoma. In another aspect, the compound is CAPE. In another aspect, the CAPE analog is GL8. In another aspect, the GL8 analog is As26.

In another aspect, the present disclosure relates to a composition for treating a patient with cancer including a therapeutically effective amount of CAPE or CAPE analog in combination with a therapeutically effective amount of a MYC regulating agent. In another aspect, the cancer is blood cancer. In another aspect, the blood cancer is selected from the group consisting of myeloma, leukemia or lymphoma. In another aspect, the compound is CAPE. In another aspect, the CAPE analog is GL8. In another aspect, the CAPE analog is As26. In another aspect, the MYC regulating agent is a BET inhibitor. In a further aspect, the BET inhibitor is a small molecule inhibitor JQ1. In another aspect, the small molecule inhibitor is MYCi361 and related analogs.

In certain embodiments, the composition is a pharmaceutical composition. In certain embodiments, the composition is a dietary supplement. In certain embodiments, the composition includes a carrier. In certain other embodiments, the carrier is a pharmaceutically acceptable carrier.

In certain aspects of the present invention, pharmaceutically acceptable compositions are provided, wherein these compositions comprise any of the compounds or a pharmaceutically acceptable salt thereof, as described herein, and optionally comprise a pharmaceutically acceptable carrier, adjuvant or vehicle. In certain embodiments, these compositions optionally further comprise one or more additional therapeutic agents.

It will also be appreciated that certain of the compounds of the present invention can exist in free form for treatment, or where appropriate, as a pharmaceutically acceptable derivative or a prodrug thereof. According to the present invention, a pharmaceutically acceptable derivative or a prodrug includes, but is not limited to, pharmaceutically acceptable salts, esters, salts of such esters, or any other adduct or derivative which upon administration to a patient in need thereof is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. A “pharmaceutically acceptable salt” means any non-toxic salt or salt of an ester of a compound of this invention that, upon administration to a recipient, is capable of providing, either directly or indirectly, a compound of this invention or an inhibitorily active metabolite or residue thereof.

In certain aspects of the present disclosure, pharmaceutically acceptable compositions of the present disclosure can be administered to humans and other animals at a unit dose within the range 0.01-2000 mg/kg, particularly 2.5-1000 mg/kg, particularly 5-500 mg/kg, particularly 0.01-100 mg/kg, particularly 0.5-50 mg/kg, and particularly 0.4-2 mg/kg, and particularly 10 mg/kg, 15 mg/kg and 20 mg/kg and this should provide a therapeutically effective dose. However, the daily dose will necessarily be varied depending upon the host treated, the particular route of administration, and the severity of the illness being treated. Accordingly, the optimum dosage may be determined by the practitioner who is treating any particular patient.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1a and FIG. 1b are heatmap images of RNA sequencing results;

FIG. 1c is a graph showing quantification of HDAC1gene expression;

FIG. 2a and FIG. 2b are Western blots of key protein targets HDAC1, Sp1, IRF4, IKZF1 and c-MYC lowered by CAPE and its closely related analogs in comparison with the HDAC inhibitor (panobinostat) in a human myeloma cell line;

FIG. 3 is a graph showing the treatment effect of panobinostat and IMiDs (Pomalidomide or Lenalidomide) in comparison with panobinostat and CAPE analogs treatment;

FIG. 3a is a graph showing combinatorial effect of As26 with HDACi panobinostat;

FIG. 4 is a graph showing the effect of another pan-HDAC inhibitor, Trichostatin A, and potent CAPE analog As26 combination;

FIG. 5 is a graph revealing the effect of the Sp1 inhibitor TMP and potent CAPE analog As26 combination;

FIG. 6 is a Western blot of a combination treatment effect of a combination of the Sp1 inhibitor Terameprocol (TMP) and the potent CAPE analog As26 on Sp1 and IRF4 protein level; and

FIG. 6a is a Western blot of a 100 uM TMP effect and IKZF1 expression.

### DETAILED DESCRIPTION

Experimental Section

Cell Culture. Human myeloma cell lines JJN3 were purchased from ATCC, KMM-1 was procured from the Japanese Collection of Research Bioresources Cell Bank (JCBR, Japan). The MM1.R cell line was a kind gift from Dr. Jonathan Keats, Translational Genomics Research Institute (TGen, USA). JJN3 cells were grown in RPMI-1640 medium (Sigma Aldrich, USA) supplemented with 5% heat inactivated FBS (Gibco), 1% I-glutamine (Gibco), 5 mM HEPES, and 1% Penstrep (Sigma Aldrich, USA); KMM-1 cells in RPMI-1640 media supplemented with 10% FBS, 1% L-glutamine (Gibco) and 1% Penstrep; MM1.R cells in advanced RPMI-1640 medium with 4% FBS and 1% Glutamax, in a humidified 5% CO₂ incubator at 37° C. Cell lines were tested regularly for mycoplasma and kept free of contamination. Lenalidomide, terameprocol (Sigma Aldrich, USA) and pomalidomide from Selleck chemicals were used.

CAPE (2) Analogs Synthesis. Analogs of CAPE were synthesized, as previously described (Sanderson et al., 2013, Selka et al., 2019). A purity of >95% has been established for all tested compounds.

Cell Growth Inhibition Studies. A 48-h cell growth inhibition assay was conducted using the PrestoBlue cell viability kit (Invitrogen, ON, Canada). Cells were plated at 10,000 cells/well in 96-well tissue culture plates. The effect of single inhibitor and inhibitor combination treatments were done using appropriate vehicle and medium (blank) controls. Cells were incubated at 37° C. in a 5% CO₂ incubator for 48 h or 72 h depending on the chosen duration of the study. At the end of the treatment, cell growth inhibition was determined by the PrestoBlue assay. In brief, 10% PrestoBlue reagent was added to untreated and treated cells in 1004 medium in each well and incubated at 37° C. for one and a half hours. Cell growth was assessed fluorometrically at 37° C. using the BioTek Synergy H₄ Hybrid Multi-Mode microplate reader (535 nm excitation; 615 nm emission) with Gen 2.06 analyses software. Fluorescence values were corrected for any background fluorescence of the medium and test fractions by subtracting fluorescence readings of the appropriate blanks from the mean fluorescence readings of the untreated control and test wells. The percentage inhibition of cell growth was then defined as 1−(mean test well fluorescence/mean control well fluorescence)×100.

Western/Immunoblotting Methods. Protein lysates were obtained from untreated and CAPE (2) analog-treated myeloma cells and the concentration of proteins was determined by the BCA method. Proteins were resolved in a 4-20% criterion gel followed by electrophoretic transfer to nitrocellulose membrane (Bio-Rad Laboratories Inc., Germany). Membranes were then blocked with 5% skimmed milk in tris-buffered saline-tween (TBS-T) for 1 h and then, after rapid TBS-T washes, incubated overnight at 4° C. with a primary antibody of interest. Goat polyclonal anti-IRF4, rabbit polyclonal anti-Ikaros (Santa Cruz Biotechnology, Santa Cruz, Calif.); mouse monoclonal anti-Caspase-8, rabbit anti-Caspase-3, cleaved Caspase-3, anti-Sp1 (Cell Signaling Technology, Whitby, ON), rabbit monoclonal anti-MYC and mouse monoclonal β-actin antibody (Abcam, Cambridge, Mass.) were the primary antibodies used for this study. Following this, the membrane was incubated with HRP conjugated secondary antibodies. Protein levels in immunoblots were detected with an Amersham™ ECL™ Prime western blotting detection kit (GE Healthcare, ON, Canada), the imaging was performed using the ChemiDoc™ MP imaging system (Bio-Rad Laboratories, USA) and densitometry analyses were done using the Image Lab 5.0 software.

RNA Seq and Bioinformatics Analyses:

RNA from myeloma cells (treated and untreated) was isolated and checked for integrity using the Agilent Bioanalyzer 2100 system (Agilent Technologies, Santa Clara, Calif., USA) and quantified using a Qubit fluorometric assay (Thermo Fisher Scientific, USA). RNA Seq was carried out at the Genome Quebec, Montreal, Canada. In brief, total RNA was quantified using a NanoDrop Spectrophotometer ND-1000 (NanoDrop Technologies, Inc.) and its integrity was assessed on a 2100 Bioanalyzer (Agilent Technologies, USA). rRNA was depleted from 250 ng of total RNA using QlAseq FastSelect (Human 96rxns). cDNA synthesis was achieved with the NEBNext RNA First Strand Synthesis and NEBNext Ultra Directional RNA Second Strand Synthesis Modules (New England BioLabs). The remaining steps of library preparation were done using the NEBNext Ultra II DNA Library Prep Kit for Illumina (New England BioLabs). Adapters and PCR primers were purchased from New England BioLabs. Libraries were quantified using the Quant-iT™ PicoGreen® dsDNA Assay Kit (Life Technologies) and the Kapa Illumina GA with Revised Primers-SYBR Fast Universal kit (Kapa Biosystems). Average size fragment was determined using a LabChip GX (PerkinElmer) instrument. All bioinformatics analyses were done by the Canadian Centre for Computational Genomics (C3G)-Montréal Node, Canada.

Experimental Results

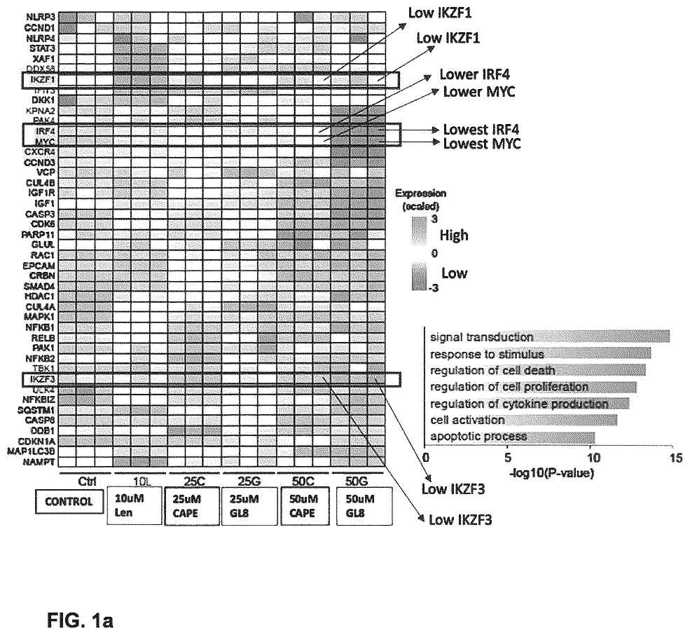

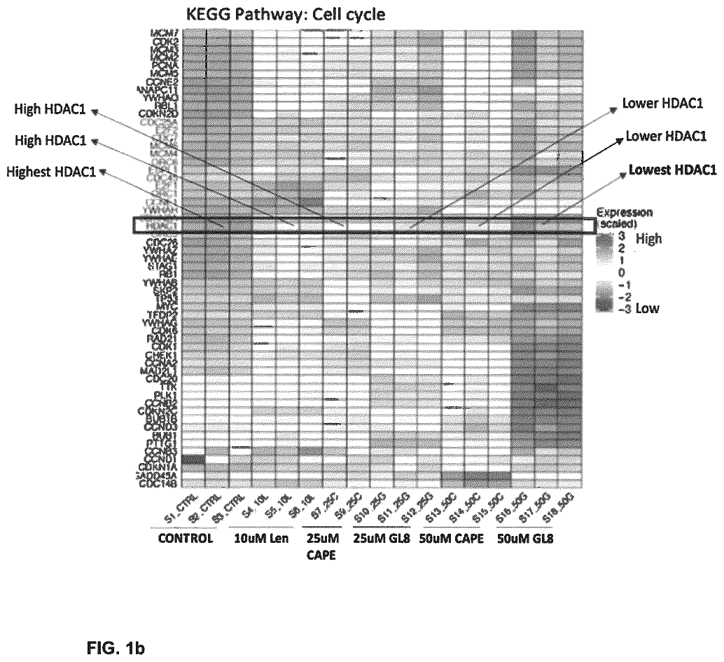

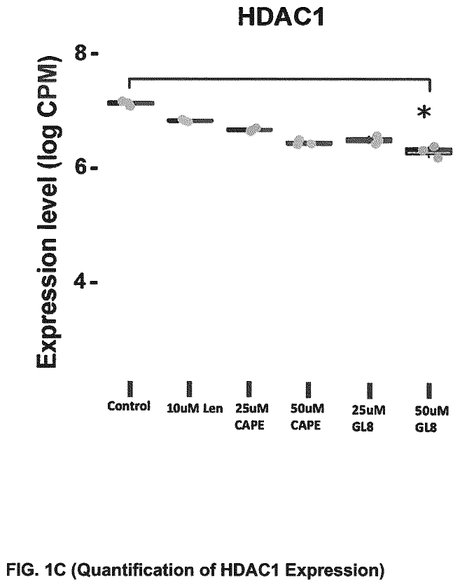

FIG. 1a, FIG. 1b and FIG. 1c are heatmap images for RNA sequence results for gene expression profiling done using RNA isolated from untreated human myeloma cells (KMM-1) and myeloma cells treated with Lenalidomide (10 uM), CAPE (25 and 50 uM) and GL8 (25 and 50 uM). The key downregulated gene targets IKZF1, IKZF3, IRF4, MYC and HDAC1 in CAPE (50 uM) and GL8 (25 uM and 50 uM) treated conditions in comparison with untreated and lenalidomide treated conditions, which are related to combination therapeutic approaches, are highlighted.

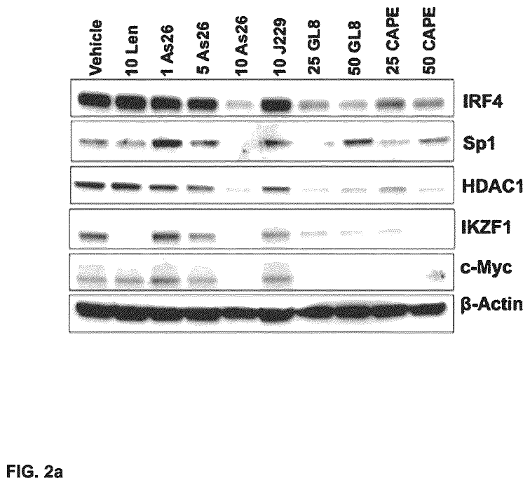

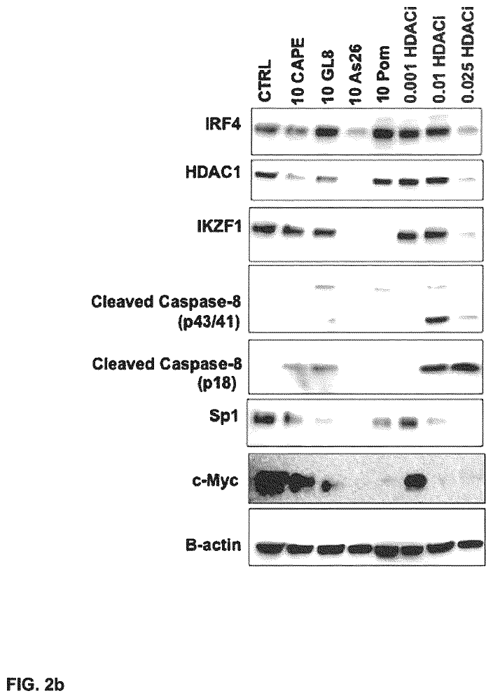

FIG. 2a and FIG. 2b are Western blots of key protein targets HDAC1, Sp1, IRF4, IKZF1 and c-MYC lowered by CAPE and its closely related analogs in comparison with the HDAC inhibitor (panobinostat) in human myeloma cell lines JJN3 and MMIR. FIG. 1a is for a cell line—JJN3; where numbers 1, 5, 10 and 25 in the figure labels depict the concentration of the compounds in uM, and FIG. 2b is for cell line—MMIR.

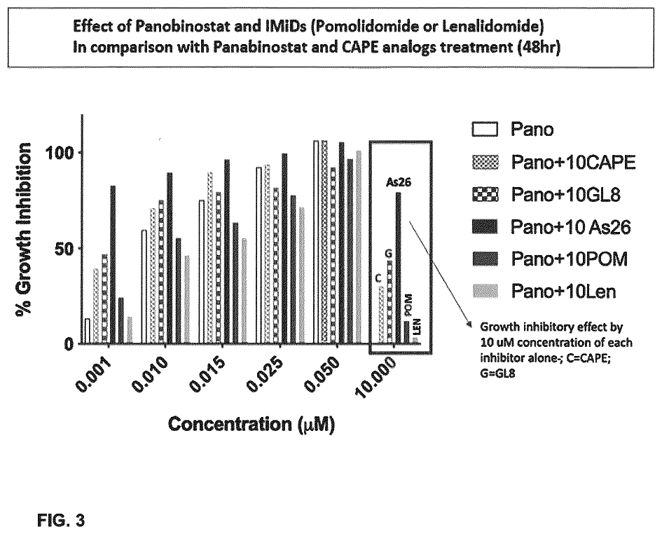

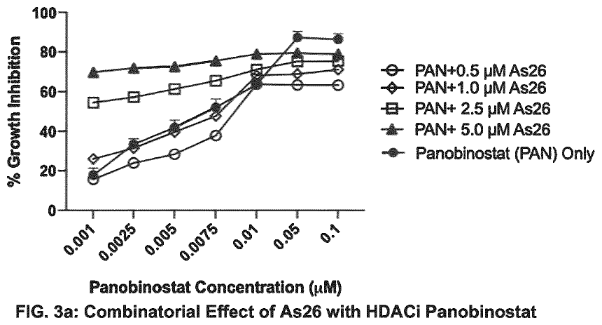

FIG. 3 is a graph revealing the treatment effect of panobinostat and IMiDs (Pomalidomide or Lenalidomide) in comparison with panobinostat and CAPE analogs combination treatment.

Referring to FIG. 3a, in the myeloma cell line JJN3, at a concentration as low as 0.001 nM, panobinostat alone demonstrated 17±3% cell growth inhibition only. However, combination treatments involving 2.5 μM As26 or 5 μM As26 with 0.001 nM panobinostat dramatically increased myeloma cell growth inhibition to 54.4% and 69.8% respectively.

Combining CAPE analog As26 with panobinostat can remarkably lower the dose of HDAC inhibitor, thereby reducing the adverse effects and resistance associated with administration of HDAC inhibitors alone.

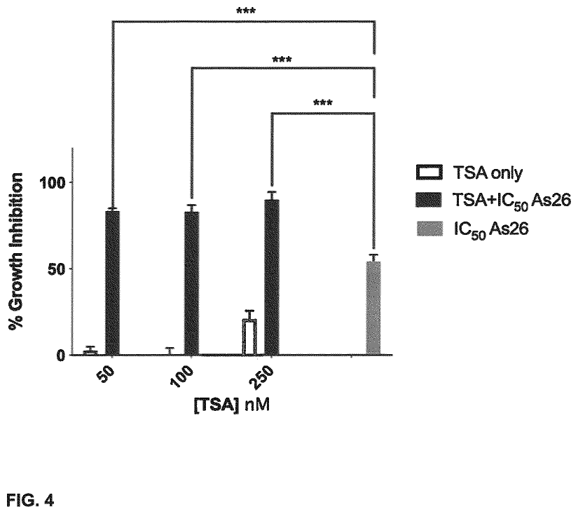

FIG. 4 is a graph showing the effect of another pan-HDAC inhibitor, Trichostatin A, IC 50 amount of potent CAPE analog As26 alone, in comparison with Trichostatin A and IC 50 As26 combination treatment. The combination was found to provide augmented myeloma cell growth inhibition.

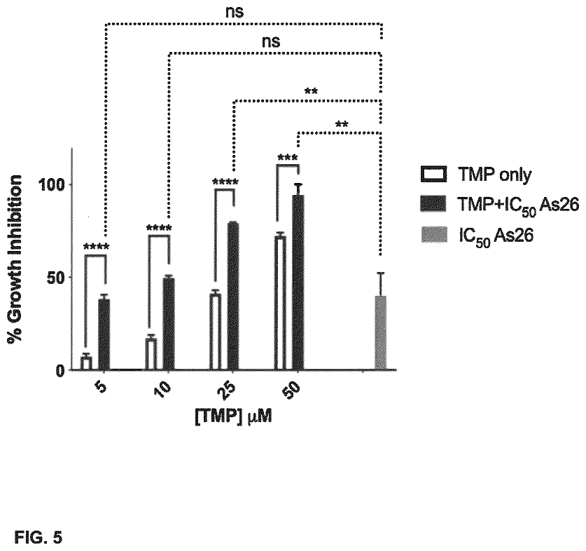

FIG. 5 is a graph showing the effect of the Sp1 inhibitor Terameprocol (TMP) and potent CAPE analog As26 combination. The combination was found to provide augmented myeloma cell growth inhibition, and

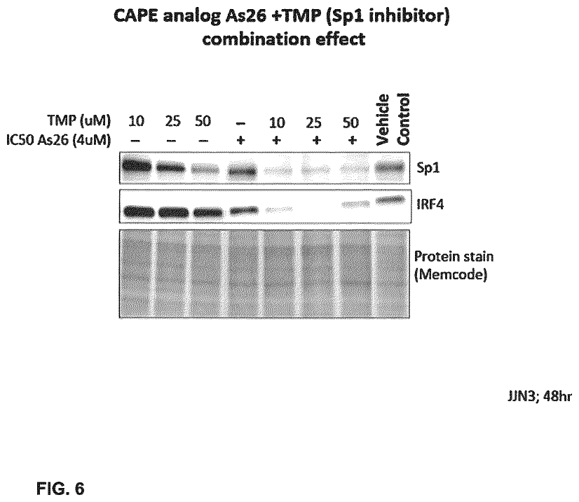

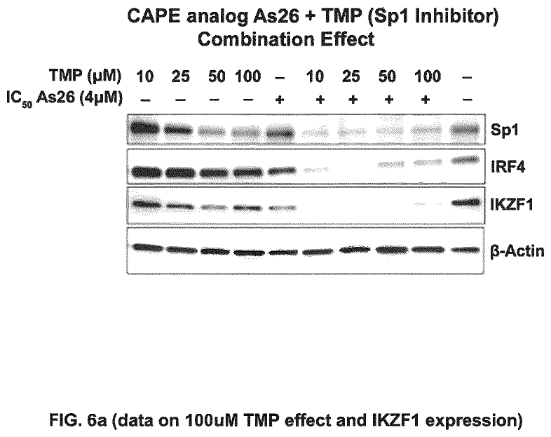

FIGS. 6 and 6a are Western blots showing the combination treatment effect of Sp1 inhibitor TMP and the potent CAPE analog As26 on Sp1 and IRF4 protein levels. The combination was found to provide augmented myeloma cell growth inhibition.

### REFERENCES

1. Patent Cooperation Treaty international application serial no. PCT/CA2018/000198 “Compositions and Methods for Inhibiting Blood Cancer Cell Growth”.
2. Mitsiades, N, Mitsiades, CS, Richardson, PG, McMullan, C, Poulaki, V, Fanourakis, G, et al. Molecular of histone deacetylase inhibition in human malignant B cells. Blood. (2003) 101:4055-62. doi: 10.1182/blood-2002-11-3514.
3. Subramanian, S, Bates, SE, Wright, JJ, Espinoza-Delgado, I, Piekarz, RL. Clinical Toxicities of Histone Deacetylase Inhibitors. Pharmaceuticals (Basel). 2010; 3(9):2751-2767. Published Aug. 26, 2010. doi:10.3390/ph3092751.
4. Bailey, H, Stenehjem, DD, Sharma, S. Panobinostat for the treatment of multiple myeloma: the evidence to date. J Blood Med. Oct. 8, 2015; 6:269-76. doi: 10.2147/JBM.S69140. PMID: 26504410; PMCID: PMC4603728.
5. Blake Marriott, J.; Muller, G.; Stirling, D.; Dalgleish, A. G. Immunotherapeutic and Antitumour Potential of Thalidomide Analogues. Expert Opin. Ther. 2001. https://doi.org/10.1517/14712598.1.4.675.
6. Lu, G., Middleton, R. E., Sun, H., Naniong, M., Ott, C. J., Mitsiades, C. S., . . . Kaelin, W. G. (2014). The Myeloma Drug Lenalidomide Promotes the Cereblon-Dependent Destruction of Ikaros Proteins. Science, 343(6168), 305-309. https://doi.org/10.1126/science.1244917.
7. Krönke, J., Udeshi, N. D., Narla, A., Grauman, P., Hurst, S. N., McConkey, M., et al. (2014). Lenalidomide Causes Selective Degradation of IKZF1 and IKZF3 in Multiple Myeloma Cells. Science, 343(6168), 301-305. https://doi.org/10.1126/science.1244851.
8. Richardson, P. G., Schlossman, R. L., Weller, E., Hideshima, T., Mitsiades C., Davies, F., et al. (2002). Immunomodulatory drug CC-5013 overcomes drug resistance and is well tolerated in patients with relapsed multiple myeloma. Blood, 100, 3063-3067.
9. Dimopoulos, M., Spencer, A., Attal, M., Prince, H., Harousseau, J., Dmoszynska, A. et al. (2007) Lenalidomide plus dexamethasone for relapsed or refractory multiple myeloma. N Engl J Med 357: 2123-2132.
10.Suske, G. The Sp-Family of Transcription Factors. Gene. 1999. https://doi.org/10.1016/S0378-1119(99)00357-1.
11. Smolewski, P. Terameprocol, a Novel Site-Specific Transcription Inhibitor with Anticancer Activity. I Drugs. 2008
12. Näär, A. M.; Ryu, S.; Tjian, R. Cofactor Requirements for Transcriptional Activation by Sp1. In Cold Spring Harbor Symposia on Quantitative Biology; 1998. https://doi.org/10.1101/sqb.1998.63.189.
13. Fulciniti, M.; Amin, S.; Nanjappa, P.; Rodig, S.; Prabhala, R.; Li, C.; Minvielle, S.; Tai, Y. T.; Tassone, P.; Avet-Loiseau, H.; et al. Significant Biological Role of Sp1 Transactivation in Multiple Myeloma. Clin. Cancer Res. 2011. https://doi.org/10.1158/1078-0432.CCR-11-1036.
14. Han, H., Jain, AD, Truica, MI, lzquierdo-Ferrer, J, Anker, JF, Lysy, B, Sagar, V, Luan, Y, Chalmers, ZR, Unno, K, Mok, H, Vatapalli, R, Yoo, YA, Rodriguez, Y, Kandela, I, Parker, JB, Chakravarti, D, Mishra, RK, Schiltz, GE, Abdulkadir, SA. Small-Molecule MYC Inhibitors Suppress Tumor Growth and Enhance Immunother apy. Cancer Cell. Nov. 11, 2019; 36(5):483-497.e15. doi: 10.1016/j.cce11.2019.10.001. Epub Oct 31, 2019.
15.Allen-Petersen, BL, Sears, RC. Mission Possible: Advances in MYC Therapeutic Targeting in Cancer. BioDrugs. 2019 October; 33(5):539-553. doi: 10.1007/s40259-019-00370-5.
16. Kato, F, et al. MYCL is a target of a BET bromodomain inhibitor, JQ1, on growth suppression efficacy in small cell lung cancer cells. Oncotarget. 2016,7(47):77378-88.
17. Doroshow, DB, Eder, JP, LoRusso, PM. BET inhibitors: a novel epigenetic approach. Ann Oncol. 2017; 28(8):1776-87.
18. Sanderson, J. T.; Clabault, H.; Patton, C.; Lassalle-Claux, G.; Jean-Francois, J.; Pare, A. F.; Hébert, M. J. G.; Surette, M. E.; Touaibia, M. Bioorg. Med. Chem. 2013, 21, 7182-7193.
19. Selka, A.; Doiron, J. A.; Lyons, P.; Dastous, S.; Chiasson, A.; Cormier, M.; Turcotte, S.; Surette, M. E.; Touaibia, M. Eur. J. Med. Chem. 2019, 179, 347-357.

1. A method for treating a patient with cancer, the method comprising:
administering to the patient a therapeutically effective amount of a compound selected from the group consisting of CAPE and a CAPE analog to downregulate a cancer gene or protein target expression, wherein administration of the CAPE or the CAPE analog reduces the number or growth of cancer cells or the tumor burden or tumour growth in the patient, thereby treating the patient.

2. The method of claim 1, further comprising a downregulating agent in combination with the compound.

3. The method of claim 2, wherein the downregulating agent is a histone deacetylase (HDAC) inhibitor, wherein administration of CAPE or CAPE analog in combination with the histone deacetylase inhibitor reduces the number of cancer cells or the tumor burden in the patient, thereby treating the patient.

4. The method of claim 3, wherein the HDAC is HDAC1.

5. The method of claim 1, wherein the cancer is a blood cancer.

6. The method of claim 5, wherein the blood cancer is selected from the group consisting of myeloma, leukemia or lymphoma.

7. (canceled)

8. The method of claim 5, wherein the compound is a CAPE analog according to the formula

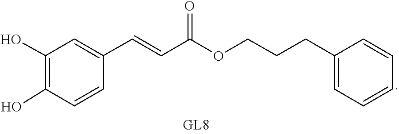 [SMILES: O=C(/C=C/c1ccc(O)c(O)c1)OCCCc1ccccc1]

9. The method of claim 5, wherein the compound is a CAPE analog according to the formula

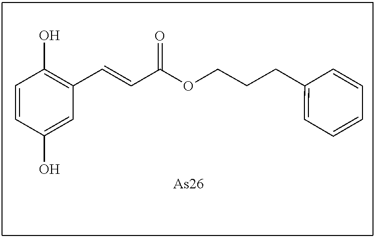

10. (canceled)

11. (canceled)

12. (canceled)

13. The method of claim 3, wherein the HDAC inhibitor is selected from the group consisting of a pan-HDAC inhibitor, panobinostat, trichostatin A, entinostat, ricolinostat, romidepsin, vorinostat, belinostat, and LAQ824.

14. The method according to claim 2, wherein the downregulating agent is an Immunomodulatory class of compounds (IMIDs), wherein administration of CAPE or CAPE analog in combination with IMiD reduces the number or growth of cancer cells or the tumor burden or tumour group in the patient, thereby treating the patient.

15. (canceled)

16. (canceled)

17. (canceled)

18. (canceled)

19. (canceled)

20. The method of claim 14, wherein the IMID is lenalidomide or pomalidomide.

21. The method of claim 2, wherein the downregulating agent is a Sp1 inhibitor, and wherein administration of CAPE or CAPE analog in combination with Sp1 inhibitor reduces the number or growth of cancer cells or the tumor burden or tumour growth in the patient, thereby treating the patient.

22. (canceled)

23. (canceled)

24. The method of claim 21, wherein the cancer is brain cancer or breast cancer.

25. (canceled)

26. (canceled)

27. (canceled)

28. The method of claim 2, wherein the downregulating agent is a MYC regulating agent, wherein administration of CAPE or CAPE analog in combination with MYC regulating agent reduces the number or growth of cancer cells or the tumor burden or tumour growth in the patient, thereby treating the patient.

29. (canceled)

30. (canceled)

31. (canceled)

32. (canceled)

33. (canceled)

34. The method of claim 28, wherein the MYC regulating agent is a BET inhibitor.

35. The method of claim 34, wherein the BET inhibitor is a small molecule inhibitor JQ1

36. The method of claim 35, wherein the small molecule inhibitor is MYCi361 and related analogs.

37. (canceled)

38. (canceled)

39. (canceled)

40. (canceled)

41. (canceled)

42. (canceled)

43. (canceled)

44. (canceled)

45. (canceled)

46. (canceled)

47. (canceled)

48. (canceled)

49. (canceled)

50. (canceled)

51. (canceled)

52. (canceled)

53. (canceled)

54. (canceled)

55. (canceled)

56. (canceled)

57. (canceled)

58. (canceled)

59. (canceled)

60. (canceled)

61. (canceled)

62. (canceled)

63. (canceled)

64. (canceled)

65. (canceled)

66. (canceled)

67. (canceled)

68. (canceled)

69. (canceled)

70. (canceled)

71. (canceled)

72. (canceled)

73. A composition for treating a patient with cancer comprising a therapeutically effective amount of CAPE or CAPE analog in combination with a therapeutically effective amount of a downregulating agent.

74. The composition of claim 73, wherein the downregulating agent is a histone deacetylase (HDAC) inhibitor.

75. The composition of claim 74, wherein the HDAC is HDAC1.

76. The composition of claim 73, wherein the cancer is blood cancer.

77. The composition of claim 76, wherein the blood cancer is selected from the group consisting of myeloma, leukemia or lymphoma.

78. (canceled)

79. The composition of claim 76, wherein the compound is a CAPE analog according to the formula:

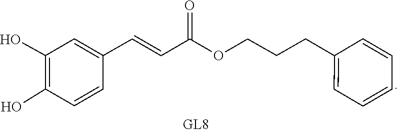 [SMILES: O=C(/C=C/c1ccc(O)c(O)c1)OCCCc1ccccc1]

80. The composition of claim 76, wherein the compound is a CAPE analog according to the formula:

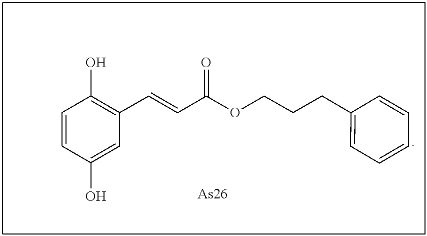

81. The composition of claim 74, wherein the HDAC inhibitor is selected from the group consisting of a pan-HDAC inhibitor, panobinostat, and trichostatin A.

82. (canceled)

83. (canceled)

84. (canceled)

85. (canceled)

86. (canceled)

87. (canceled)

88. (canceled)

89. (canceled)

90. The composition of claim 73, wherein the downregulating agent is a Sp1 inhibitor.

91. (canceled)

92. (canceled)

93. The composition of claim 90, wherein the cancer is brain cancer or breast cancer.

94. (canceled)

95. (canceled)

96. (canceled)

97. The composition of claim 74, wherein the downregulating agent is a MYC regulating agent.

98. (canceled)

99. (canceled)

100. (canceled)

101. (canceled)

102. (canceled)

103. The composition of claim 97, wherein the MYC regulating agent is a BET inhibitor.

104. The composition of claim 103, wherein the BET inhibitor is a small molecule inhibitor JQ1.

105. The composition of claim 104, wherein the small molecule inhibitor is MYCi361 and related analogs.
